(19) United States
(12) Patent Application Publication
(10) Pub. No.: US 2007/0027420 A1
(43) Pub. Date: Feb. 1, 2007

(54) Structure of ankle protection kit

(75) Inventor: Tony Yu, Taipei County (TW)

Correspondence Address: BACON & THOMAS, PLLC, 625 SLATERS LANE, FOURTH FLOOR, ALEXANDRIA, VA, 22314, US

(73) Assignee: Special Protectors Co., Ltd, Taipei County (TW)

(21) Appl. No.: 11/190,974

(22) Filed: Jul. 28, 2005

(51) Int. Cl. A61F 5/00; A61F 5/37

(52) U.S. Cl. 602/27; 128/882

(57) ABSTRACT

A device of a protection kit especially applied to an ankle, the ankle protection kit has a supporting portion from which a set of mutually opposite fixed portions extend and are pivotally connected therewith; the outer sides of the fixed portions are fixed by means of fixing tapes, so that the entire ankle protection kit can be fixed on the ankle. The structure is characterized in that: the fixing tapes has thereon a male magic tape section, a female magic tape section and an elastic section, the fixing tapes can have an effect of elastically extending by having the elastic section.

### BACKGROUND OF THE INVENTION

1. Field of the Invention

The present invention is related to a device of a protection kit, and especially to a protection kit applied to an ankle.

2. Description of the Prior Art

Human joints of bones are subjected to getting ill and feeling pains after activities or exercises for long times; there by during the stage of rehabilitation, most patients choose to wear on protection kits to avoid large amount of moving of the injured body areas, especially the injured joints.

Particularly, joints at the ankles of people's bodies are in the state of exercising for long times every day by walking; thus ankle extensions have been developed for effective reducing large amount of exercises of their ankles.

The protection kits used generally for ankles presently mostly are integrally formed; alternatively, they are assembled each using three pieces of fixing plates, the protection kits then are fixed at the areas of ankles directly by means of magic tapes, thus the ankles can be fixed to avoid large amount of exercises of the ankles.

However, a conventional protection kit mainly is fixed on an ankle by means of magic tapes; when it is fixed on the ankle, no surplus space is left, this may result discomfort of wearing and is subjected to making suffering of the ankle.

By virtue that a magic tape is used to directly and firmly fix a protection kit on an ankle, and the ankle is subjected to being bound tight; when the protection kit is unduly worn, it not only can lose its original function, but also can make suffering of the ankle and make an injured area more seriously damaged.

### SUMMARY OF THE INVENTION

In view of the above stated problems, the inventor of the present invention provides a structure of ankle protection kit based on his professional experience of years in studying, designing and manufacturing same kind of products and after dedicating of his energy with hard study and developing.

Thereby, the structure of ankle protection kit of the present invention has a supporting portion from which a set of mutually opposite fixed portions extend and are pivotally connected therewith; the inner sides of the fixed portions are provided with mutually opposite air cushions, and the outer sides of the fixed portions are fixed by means of fixing tapes, so that the entire ankle protection kit can be fixed on the ankle. The structure is characterized in that: the fixing tapes has thereon a male magic tape section, a female magic tape section and an elastic section, the fixing tapes can have an effect of elastically extending by having the elastic section.

And more, the fixed portions and the air cushions are provided thereon respectively with male magic tapes and female magic tapes, and the air cushions on the fixed portions can be detached for changing, a user can change them for more suitable air cushions by himself. The male magic tapes provided on the fixed portions exactly cover pivot joints of the fixed portions and the supporting portion, thus friction of the air cushions against the pivot joints can be avoided to ensure the integrity of the air cushions.

Moreover, the back side of the bottom of the supporting portion is designed to be a coarse surface to increase the friction force of the back side of the bottom of the supporting portion and to get an effect of slide proofing.

The present invention will be apparent in its content and the effect to be achieved after reading the detailed description of the preferred embodiment thereof in reference to the accompanying drawings.

### BRIEF DESCRIPTION OF THE DRAWINGS

FIG. 1 is a perspective schematic view of the present invention;

FIG. 2 is a first anatomic perspective view of the present invention;

FIG. 3 is a second anatomic perspective view of the present invention.

### DETAILED DESCRIPTION OF THE PREFERRED EMBODIMENT

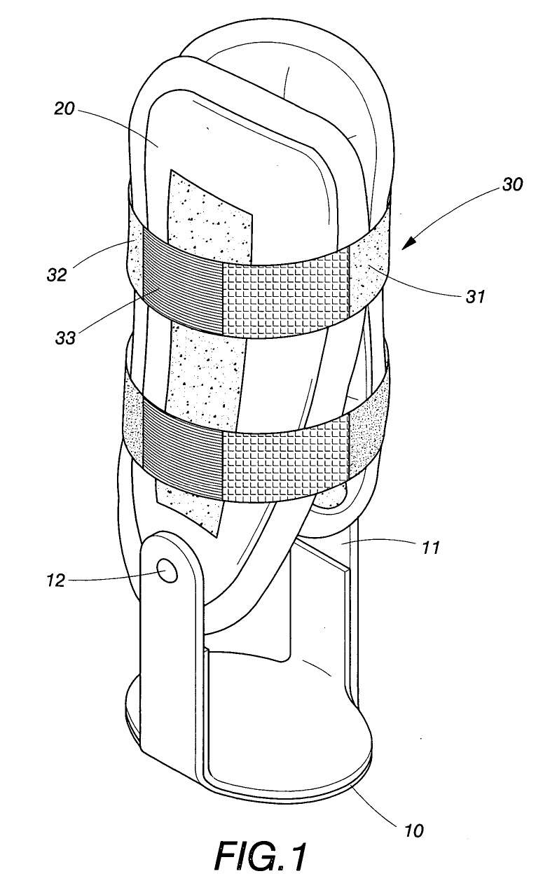

Referring firstly to FIG. 1, in the structure of ankle protection kit of the present invention, the ankle protection kit mainly comprises a supporting portion 10 braced on the sole of a foot, a set of mutually opposite fixed portions 20 fixed on lateral sides of an ankle and at least a fixing tape 30 wrapped around the fixed portions 20; wherein the fixed portions 20 are pivotally connected with tailing ends of two pivot connecting arms 11 on two lateral sides of the supporting portion 10, and can be pivoted on the tailing ends of the two pivot connecting arms 11 by means of pivot joints 12; and the fixing tape 30 able to be wrapped around the fixed portions 20 renders the entire ankle protection kit to be fixed on the ankle. The structure is characterized in that:

The fixing tape 30 further has thereon a male magic tape section 31, a female magic tape section 32 and an elastic section 33; when the fixing tapes 31 are wrapped around the fixed portions 20, and the fixed portions 20 are fixed on the two lateral sides of the ankle, an effect of elastically extending can be obtained by having the elastic section 33, this can increase coziness of wearing the ankle protection kit.

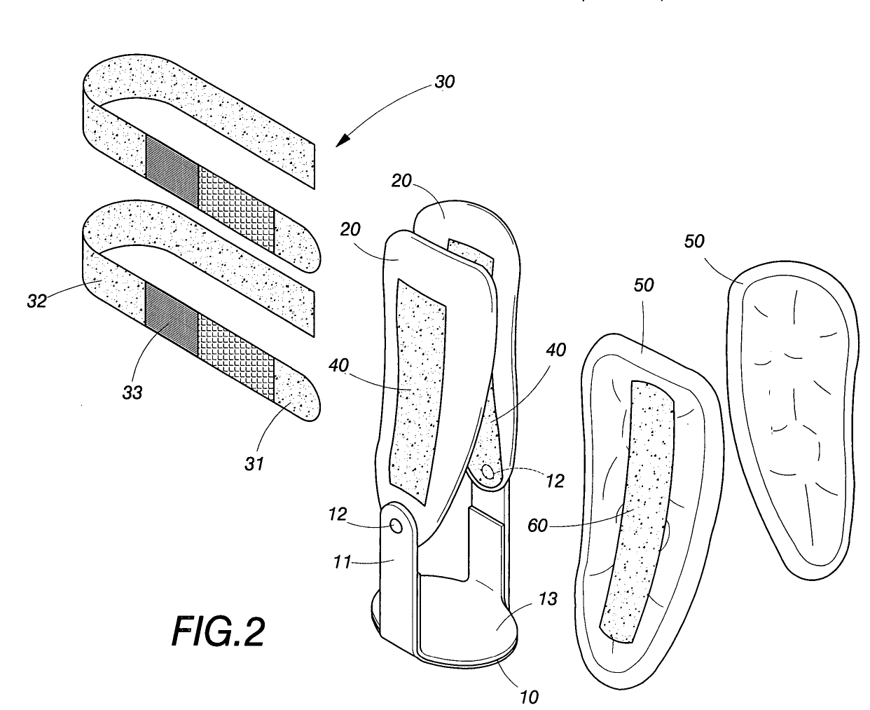

As shown in FIG. 2, each of the two lateral sides of the supporting portion 10 has a pivot connecting arm 11 extending outwards, and the supporting portion 10 is further provided on the upper surface thereof with a buffering member 13 to absorb the pressure exerted by the sole of the foot, this in turn avoid direct force exerting of the sole of the foot on the supporting portion 10. The fixed portions 20 are pivotally connected with tailing ends of two pivot connecting arms 11, and the inner as well as the outer sides of each of the fixed portions 20 both are provided with male magic tapes 40, and are further provided with air cushions 50. The air cushions 50 are positioned on the inner sides of the fixed portions 20 by the sticking function between the male magic tapes 40 on the inner sides of the fixed portions 20 and female magic tapes 60 provided on the air cushions 50 and being in opposition to the male magic tapes 40.

And the male magic tapes 40 provided on the inner sides of the fixed portions 20 are used to cover the pivot joints 12 between the fixed portions 20 and the two pivot connecting arms 11 in order to avoid abrading of the pivot joints 12 directly against the air cushions 50, this can keep the integrity of the air cushions 50, and also can elongate the life of use of the air cushions 50.

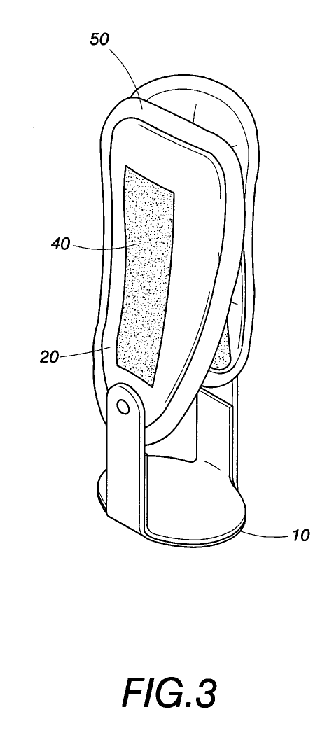

Referring to FIG. 3, the air cushions 50 mainly are positioned on the inner sides of the fixed portions 20 by the sticking function between the male magic tapes 40 with the female magic tapes 60. Thereby the air cushions 50 can be detached freely from the inner sides of the fixed portions 20 for changing to allow a user to put on air cushions 50 that he thinks suitable; and the entire ankle can feel cozy.

And more, by virtue that the supporting portion 10 is used to be put on the sole of the foot, the back side of the bottom of the supporting portion 10 is designed to be a coarse surface to increase the friction force of the back side of the bottom of the supporting portion 10; and when the ankle protection kit, an effect of slide proofing can be obtained. This avoids that the back side of the bottom of the supporting portion 10 slides when the user walks, and thereby safety of wearing the ankle protection kit can be increased.

The embodiment given is only for illustrating the present invention, and not for giving any limitation to the scope of the present invention. It will be apparent to those skilled in this art that various equivalent modifications or changes without departing from the spirit of this invention shall also fall within the scope of the appended claims.

1. A structure of ankle protection kit, said structure mainly comprises a supporting portion braced on the sole of a foot, a set of mutually opposite fixed portions fixed on two lateral sides of an ankle and at least a fixing tape wrapped around said fixed portions; wherein said fixed portions are pivotally connected with tailing ends of two pivot connecting arms on two lateral sides of said supporting portion, and said fixing tape wrapped around said fixed portions renders said ankle protection kit to be fixed on said ankle; said structure is characterized in that:
said fixing tape has thereon a male magic tape section, a female magic tape section and an elastic section; when said fixing tapes are wrapped around said fixed portions, and said fixed portions are fixed on two lateral sides of said ankle, an effect of elastically extending is obtained by having said elastic section, this increases coziness of wearing said ankle protection kit.

2. The structure of ankle protection kit as in claim 1, wherein: said supporting portion is further provided on an upper surface thereof with a buffering member to absorb pressure exerted by said sole of said foot.

3. The structure of ankle protection kit as in claim 1, wherein: an inner as well as an outer side of each of said fixed portions both are provided with male magic tapes, and are further provided with air cushions, said air cushions are positioned on said inner sides of said fixed portions by sticking function between said male magic tapes on said inner sides of said fixed portions and female magic tapes provided on said air cushions and being in opposition to said male magic tapes; and said air cushions are detached freely from said inner sides of said fixed portions for changing.

4. The structure of ankle protection kit as in claim 3, wherein: said male magic tapes provided on said inner sides of said fixed portions are used to cover pivot joints between said fixed portions and said two pivot connecting arms, in order to avoid abrading of said pivot joints directly against said air cushions.

5. The structure of ankle protection kit as in claim 1, wherein: a back side of a bottom of said supporting portion is designed to be a coarse surface to increase friction force of said back side of said bottom of said supporting portion.
